(19) United States
(12) Patent Application Publication
(10) Pub. No.: US 2003/0100082 A1
(43) Pub. Date: May 29, 2003

(54) Methods for isolation of proanthocyanidins from flavonoid-producing cell culture

(76) Inventors: Mary Ann Lila, Urbana, IL (US); David S. Seigler, Urbana, IL (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/280,856

(22) Filed: Oct. 25, 2002

Related U.S. Application Data
(60) Provisional application No. 60/356,858, filed on Feb. 13, 2002.
(60) Provisional application No. 60/336,368, filed on Oct. 31, 2001.

(51) Int. Cl. C12P 17/06; C12N 5/04; C07D 311/32

(52) U.S. Cl. 435/125; 435/419; 549/403

(57) ABSTRACT

A method for isolation of proanthocyanidins from flavonoid-producing cell cultures is disclosed. More specifically, the invention relates to the isolation of catechin, epicatechin, proanthocyanidin B-2, and other proanthocyanidins from Vaccinium pahalae Skottsberg cultures. The invention also provides a method for modifying the content of proanthocyanidins in a flavonoid-producing culture. Further, the invention relates to a method of performing metabolic studies with proanthocyanidins.

### CROSS REFERENCE TO RELATED APPLICATIONS

This application claims priority from U.S. provisional applications Serial Nos. 60/336,368 and 60/356,858, filed Oct. 31, 2001 and Feb. 13, 2002, respectively, both of which are incorporated herein by reference in their entirety.

### FIELD OF THE INVENTION

The present invention relates to a method of isolating proanthocyanidins from flavonoid-producing cell cultures. More specifically, the invention relates to the isolation of catechin, epicatechin, proanthocyanidin B-2, and other proanthocyanidins from Vaccinium pahalae Skottsberg cultures. The method of proanthocyanidin isolation comprises establishing a pigmented cell culture, performing a cell culture extraction to isolate proanthocyanidins, and fractionating isolated proanthocyanidins by vacuum chromatography. The invention also provides a method of modifying the content of proanthocyanidins in a flavonoid-producing cell culture. Preferably, the content of proanthocyanidins is increased, thereby increasing the antioxidant and anticarcinogenic capacity of proanthocyanidins. Further, the invention relates to a method of performing metabolic fate studies with proanthocyanidins. This method utilizes ¹⁴C-labeled proanthocyanidins isolated by the method of the present invention to measure metabolic rates and/or fates of the labeled compounds.

### BACKGROUND OF THE INVENTION

Polyphenolic compounds, which are greatly represented in edible plants, have recently attracted great interest due to their antioxidant properties. The evidence for chemopreventive, anti-inflammatory, and cardioprotective roles of these phytochemicals from teas, red wines, and fruits is rapidly growing (Aherne and O'Brian, 1999; Gottrand et al., 1999; Koganov et al., 1999). However, while the antioxidant properties of dietary constituents such as vitamins E and C are well understood, it is not clear through what mechanisms polyphenolic compounds exert their antioxidant effects.

Bioflavonoids belong to the family of polyphenolic compounds, and include various subclasses such as flavans, flavanones, flavones, anthocyanins, and proanthocyanidins (also referred to as “condensed tannins”). Proanthocyanidins (colorless compounds) and anthocyanins (pigmented compounds) are considered by many to be the key active flavonoid compounds in teas, red wines, and fruits (Colatuoni et al., 1991; Castonguay et al., 1997; Das et al., 1999; Koga et al., 1999; Narayan et al., 1999). In addition to being present in beverages of botanical origin, the flavonoids are also found in all aerial parts of plants, with high concentrations in the skin, bark, and seeds.

The difficulties associated with isolation of flavonoid compounds from plants have so far been the culprits of slow progress in understanding the biology of these important compounds. There have been numerous reports in the literature relating to tannins from plant cultures, however most of these reports were based on detecting the presence of tannins by histochemical or colorimetric assessment.

The use of HPLC technology in recent years has allowed for improved isolation of proanthocyanidins from plant sources, however their isolation still presents a daunting task. Isolation of polyphenols from plant sources is in general complicated by the presence of other plant substances, mainly polysaccharides, which can interfere with polyphenol isolation. Furthermore, polysaccharides can reduce the activity of target compounds in bioactivity assays (Ferreira et al., 1999), making it more difficult to establish the physiological functions of the tested compounds. For example, pectins and sugars from fruits reduce the efficiency and increase the time necessary for chromatographic isolation of flavonoids, and in addition, pectins may interfere with determining the results of standard laboratory assays for antioxidant capacity. Flavonoids may also be partially degraded, escape detection, and/or become inactive in the course of many standard laboratory extraction/fractionation procedures (Porter, 1993).

Plant cell cultures have recently started to play a role in isolation of bioflavonoids. In case of proanthocyanidins, several groups were able to isolate particular substances belonging to this family from cultures. For instance, C(4)-C(8) linked (−)-epicatechin-(+)-catechin and gallic acid have been isolated from a Rosa culture (Muhitch and Fletcher, 1984). Suspension cultures and calluses of Cryptomeria japonica (L.f.) D. Don were found to produce as much as 26% of dry weight as procyanidins (Teramoto and Ishikura, 1985; Ishikura and Teramoto, 1983), and Pseudotsuga menziesii (Mirb.) Franco suspension cultures as much as 40% of their dry weight as procyanidins (Stafford and Cheng, 1980). Cultures of Ginkgo biloba L. were reported to accumulate as much as 50-60% of their dry weight as procyanidins and prodelphinidins (Stafford et al., 1986). The tannins that occurred in calli of the legumes Onobrychis viciifolia and Lotus corniculatus had not been identified or quantified (Lees, 1986). In the reports published so far and relating to proanthocyanidins, these compounds were either not characterized or only narrow ranges of proanthocyanidins had been reported. Furthermore, none of the reports addressed the issue of bioactivity of these compounds.

Learning more about proanthocyanidin bioactivity could play a role in developing new therapeutics due to the fact that flavonoids have been recognized for many valuable medicinal properties such as antioxidant, anti-inflammatory, antispasmodic, antihistaminic, peripheral vasodilatory, platelet antiaggregating, vasoprotective in terms of altered capillary fragility and permeability, and antiallergic. See U.S. Pat. No. 5,043,323. These properties stem from the ability of flavonoids to scavenge free radicals and interfere with enzyme systems involving enzymes such as phosphodiesterase, lipooxygenase, cyclooxygenase, aldosereductase, and histidine-decarboxylase. See U.S. Pat. No. 5,043,323.

Isolation of several proanthocyanidins from Vaccinium species, specifically Vaccinium vitis-idaea L. has been reported (Weinges et al., 1968; Thompson et al., 1972). These include proanthocyanidin B-1 [(−)-epicatechin-(4β→8)-(−)-epicatechin]; B-2 [(−)-epicatechin-(4β→8)-(−)-epicatechin]; B-3 (+)-catechin-(4β→8)-(+)-catechin]; and B-7 [(−)-epicatechin-(4β→6)-(+)-catechin]. These compounds occurred in amounts ranging from 0.05% to 0.1% of freshly obtained, unripe tissue (Thompson et al., 1972), indicating low yield of proanthocyanidins from these plant tissues. Some of the proanthocyanidins have also been detected in Vaccinium cell cultures including V. pahalae (Madhavi et al., 1995, 1998), but these mixtures of proanthocyanidins have not been separated or characterized.

Accordingly, a need exists to identify methods that allow for improved isolation and characterization of proanthocyanidins, wherein the improved isolation constitutes simplified isolation procedures with better yield of desired substances. In particular, bioactive proanthocyanidins such as those from the genus Vaccinium require further elucidation.

### SUMMARY OF THE INVENTION

Accordingly, among the objects of the present invention is the provision of methods for isolation of proanthocyanidins from flavonoid-producing cell cultures. As devised by the applicants, the methods allow for rapid, efficient and prolific isolation of a wide range of polyphenolic compounds, particularly rich in proanthocyanidins.

Briefly, the method for isolation of proanthocyanidins from a flavonoid-producing cell culture comprises initiating such culture, establishing a pigmented cell culture, extracting the proanthocyanidins from the pigmented cell culture, and fractionating the proanthocyanidins by vacuum chromatography.

Typically, the flavonoid-producing cell culture is initiated from a stable, continuous shoot microculture of an ericaceous plant. Preferably, the plant belongs to the Vaccinium family, and more preferably the plant is Vaccinium pahalae Skottsberg (also known as ohelo). The pigmented suspension culture is next established by transferring the initiated culture from the maintenance medium, incubated in the dark to a color-inducing medium, incubated under the appropriate lighting. Once the pigmented cultures are established, the extraction of proanthocyanidins may be routinely performed on a two week rotation. Briefly, the extraction involves the following steps: filtering and extracting cell cultures with 70% acetone, removing acetone under vacuum and freeze-drying the resulting solution to yield a red solid, mixing the solid with silica gel and running it over a column, eluting the column, concentrating the eluate under vacuum and lyophilizing the concentrate. The extracted material is next subjected to fractionation in order to isolate fractions with proanthocyanidins. The fractionation of subfractions is performed by additional chromatography and monitored by TLC.

The method for isolating proanthocyanidins as described herein may contain an additional step of identifying the fractionated proanthocyanidins. Preferably, the identification of these substances is achieved through the use of ¹H-NMR, ¹³C-NMR, and MS. In another preferred aspect, the proanthocyanidins that are fractionated and identified are proanthocyanidin B-2, catechin, and epicatechin, and more preferably the isolated proanthocyanidin is proanthocyanidin B-2.

The present invention also encompasses a method for modifying the content of proanthocyanidins in a flavonoid-producing cell culture. Briefly, the method involves initiating the flavonoid-producing cell culture under conditions sufficient to allow such initiation, establishing a pigmented culture under conditions sufficient to allow the establishment of the pigmented culture, and expanding the pigmented cell culture prior to the isolation of proanthocyanidins. Thus, by modifying any of the said conditions, one can achieve a different content of proanthocyanidins in a flavonoid-producing cell culture. Preferably, the content of the proanthocyanidins is increased. In another preferred embodiment, modifying the content of proanthocyanidins is done in such way as to increase the anti-oxidant capacity of said proanthocyanidins, and even more preferably, it is their anti-carcinogenic capacity that is increased. In another preferred aspect, the flavonoid-producing cell culture comprises a Vaccinium cell culture. Even more preferably, the flavonoid-producing cell culture is a Vaccinium pahalae cell culture.

The proanthocyanidins whose content has been modified may include proanthocyanidin B-2, catechin, and epicatechin. Preferably, the content of proanthocyanidin B-2 has been modified, and even more preferably its content has been increased in the flavonoid-producing cell culture.

The invention also provides a method of performing metabolic rate/fate studies, wherein said method comprises co-incubating a flavonoid-producing cell culture with ¹⁴C-labeled precursors, thereby allowing for labeled proanthocyanidins to be produced; extracting the labeled proanthocyanidins and fractionating them by vacuum chromatography; administering a desired labeled proanthocyanidin to an animal; and measuring the uptake of the labeled proanthocyanidin by the cells of said animal and/or identifying the metabolic products of the labeled proanthocyanidin in said animal. Preferably, the uptake is measured by liquid scintillation counting, and the identification is performed by mass spectrometry analysis.

In one embodiment, the desired proanthocyanidin for metabolic studies comprises proanthocyanidin B-2, catechin, and epicatechin, and more preferably the desired proanthocyanidin comprises proanthocyanidin B-2.

Other objects and features will be in part apparent and in part pointed out hereinafter.

### BRIEF DESCRIPTION OF FIGURES

FIG. 1 is a flow chart illustrating the sequence of fractionations for a 70% acetone extract from a Vaccinium pahalae cell culture.

FIG. 2(a) is a composite drawing of TLC of a 70% acetone extract of Vaccinium pahalae cell culture.

FIG. 2(b) is a composite drawing of TLC of subfractions from chromatographic fractionation of fractions 6-9 from vacuum chromatography of fraction 12 of the extract (refer to FIG. 1 for the composition of fraction 12).

FIG. 2(c) is a composite drawing of TLC of subfractions from chromatographic fractionation of fractions 32 and 33 from fractions 6-9 above (refer to FIG. 1 for composition of fractions).

FIG. 2(d) is a composite drawing of subfractions from chromatographic fractionation of fractions 6-13 from fractionation of fractions 32 and 33 above (refer to FIG. 1 for composition of fractions).

FIG. 3 is a ¹H, ¹H-correlation spectroscopy spectrum of protons 2, 3, and 4 of a mixture of (−)-epicatechin and (+)-catechin isolated from Vaccinium pahalae cell culture.

FIG. 4 is a MALDI mass spectrum (positive ion) for subfraction 8 of fraction 12 from Vaccinium pahalae cell culture, containing a series of proanthocyanidins (A-type) ranging from trimers to heptamers.

FIG. 5 is a graph depicting the percentage inhibition of ornithine decarboxylase (ODC) activity induced by extract from Vaccinium pahalae (ohelo) cell culture extracts. Values are the mean of quadruplicate determinations; bars indicate standard error.

TABLE 1 is a table listing the effective concentrations needed to quench the galvinoxyl radical (EC_{first}), rate constants, and half-times (t_{1/2}) for ohelo cell culture extracts and various proanthocyanidin-rich fruit or seed extracts. Rate constants represent average determinations of triplicate measurements; variance: ±10%.

### ABBREVIATIONS AND DEFINITIONS

To facilitate understanding of the invention, a number of terms are defined below:

“HPLC” is the abbreviation for high pressure liquid chromatography.

“TLC” is the abbreviation for thin layer chromatography.

“MS” is the abbreviation for mass spectrometry.

“NMR” is the abbreviation for nuclear magnetic resonance.

“ODC” is the abbreviation for ornithine decarboxylase.

The term “isolation” is used herein to refer to a process by which a material is made substantially free from components that normally accompany it in its native state.

As used herein, the term “proanthocyanidins” includes proanthocyanidin monomers and oligomers.

### DETAILED DESCRIPTION OF THE INVENTION

In accordance with the present invention, applicants have discovered an efficient method for isolation of proanthocyanidins from cell cultures. Specifically, Vaccinium pahalae germplasm was established in a continuous batch culture system which allowed routine isolation of proanthocyanidins on a two week rotation. Moreover, the adaptation of Vaccinium pahalae has resulted in a prolific cell culture source of proanthocyanidins. Among the advantages provided by the applicants' method for isolation of proanthocyanidins are the following:

1) reliable, simple, and predictable isolation due to the fact that climatic conditions are controlled;

2) rapid and efficient isolation due to the simple processing that minimizes degradation of proanthocyanidins during the separation process;

3) proanthocyanidins isolated from cell culture parallel those isolated from fruits;

4) the ability to manipulate and optimize cell culture proanthocyanidin profile;

5) lack of interfering compounds such as pectins, enzymes, polysaccharides which are present in fruits and absent in applicants' cell culture.

In addition to the above mentioned benefits, proanthocyanidins isolated by the methods of the present invention can be labeled and utilized in metabolic studies.

Accordingly, the present invention provides a method for isolation of proanthocyanidins from flavonoid-producing cell cultures. This method may further include a step of identifying the isolated proanthocyanidins. The invention also encompasses methods for modifying, and particularly increasing the content of proanthocyanidins in a flavonoid-producing cell culture. In addition, methods for performing metabolic rate/fate studies are provided. These methods may be helpful in characterizing the intake and/or metabolic products of proanthocyanidins isolated according to the methods described herein. Furthermore, the proanthocyanidins isolated according to the methods of the present invention may be used for therapeutic purposes due to their known antioxidant and anti-carcinogenic properties.

The method of isolating proanthocyanidins from flavonoid-producing cell cultures consists of the following steps:

(a) initiating the flavonoid-producing cell culture;

(b) establishing a pigmented cell culture by utilizing the culture from (a);

(c) extracting the proanthocyanidins from the pigmented cell culture; and

(d) fractionating the proanthocyanidins by vacuum chromatography.

This process is generally outlined below, and a detailed protocol can be found in Example 1. Briefly, although the following specifics may be varied by those skilled in this art according to the known variations, in a preferred method, initiation of the flavonoid-producing cell cultures is achieved by setting up callus and suspension cultures from stable, continuous shoot microcultures of V. pahalae according to the protocols established by Smith et al. (1997). The cultures are maintained in fresh maintenance suspension medium and incubated in the dark. Pigmented suspension cultures may be established by transferring 4-6 ml packed cell volume from dark-incubated suspension cultures into color-induction suspension medium, and incubated under 100 μmol⁻² s⁻¹ irradiance provided by cool white fluorescent lamps. Cell cultures are subcultured at 12-14 day intervals. Following expansion, the cell cultures (≈500 ml volumes) are filtered and extracted with 70% acetone. The acetone is then removed under vacuum, and the remaining solution is freeze-dried to yield a red solid. The solid is mixed with silica gel 60, air-dried, and the mixture is then loaded onto a column with silica gel 60 that had been washed with petroleum ether. Following the loading of the mixture, the column is washed again with petroleum ether. The fractions are then eluted from the column using ethyl acetate (solvent A) and methanol-water (solvent B). At this point, all colored materials are removed from the column, and 22 fractions are collected. These fractions are concentrated under vacuum to remove volatile solvents, and water is removed by subsequent lyophilization.

Several of the resulting fractions are fractionated by additional chromatography on either silica gel or Sephadex LH-20. Each step of the fractionation is monitored by TLC on silica gel plates with ethyl acetate-methanol-water (79:11:10), using vanillin-HCl reagent and with dichromate solution followed by heating at 100° C. in each instance. Another plate is sprayed with FeCl₃ reagent. Further fractionation and purification of subfractions are accomplished by repeating the procedure, but varying solvent composition to achieve optimal separations. Any needed adjustments for any of the above-mentioned techniques can be readily determined by one skilled in the art.

The method for isolation of proanthocyanidins from cell culture may also include an additional step of identifying the isolated proanthocyanidins. Accordingly, following the above-mentioned fractionations, the structures and molecular weights of several individual compounds and the general composition of unresolved fractions may be determined, preferably by ¹H-NMR, ¹³C-NMR, and MS.

In another preferred embodiment, the isolated and identified proanthocyanidins include proanthocyanidin B-2, catechin, and epicatechin. (−)-Epicatechin and (+)-catechin were major components of fractions 4-6, and were also found in smaller amounts in fractions 7-10. These two flavan-3-ols occurred in approximate 6:1 ratio as judged by both the ¹H-NMR and ¹³C-NMR spectra. More preferably, the isolated proanthocyanidin comprises proanthocyanidin B-2, which at present is not commercially available. In the experiments outlined in Example 1, this proanthocyanidin was found in fractions 7-10, 12 and 13, as confirmed by ¹H-NMR and ¹³C-NMR data. Furthermore, the present invention encompasses additional proanthocyanidins that can be isolated by the methods described herein. For example, the visualization of TLC plates with FeCl₃ indicated the presence of phenolic materials in all but the first 3 fractions (see Example 1). Based on the TLC data, there appear to be about 20 total flavan3-ols and proanthocyanidins in fractions 4-22, of which at least 14 occur in fraction 12, the most diverse fraction. Fractions 6-18 also appear to consist primarily of flavan-3-ols and proanthocyanidins.

In another preferred aspect, the flavonoid-producing cell culture is a Vaccinium cell culture, and more preferably it is the Vaccinium pahalae cell culture. It is believed that Vaccinium pahalae cell culture is the most prolific source to date of a wide range of polyphenolic compounds, with a particular proanthocyanidin-rich fraction. In addition, the ease of applicants' method of growing V. pahalae cultures under monitored conditions and the lack of interfering substances such as polysaccharides make this plant culture particularly suitable for isolation of proanthocyanidins.

The invention also provides a method for modifying the content of proanthocyanidins in a flavonoid-producing culture. In one preferred embodiment, the conditions are varied in such a way as to increase the content of proanthocyanidins in the flavonoid-producing culture. The increase in content can be determined by performing TLC, or by using identification techniques such as ¹H-NMR, ¹³C-NMR, and MS. In yet another preferred embodiment, modifying the content of the proanthocyanidins in a flavonoid-producing culture increases the anti-oxidant capacity of said proanthocyanidins. The anti-oxidant capacity may be determined by performing a galvinoxyl free radical quenching test (see Example 2). It is to be understood that increased anti-oxidant capacity results in increased anti-carcinogenic capacity, due to the fact that antioxidants are known as potent tumor inhibitors. For instance, a test that can be applied to assess a tumor inhibitory potential of a compound is an ornithine decarboxylase assay (ODC) which determines the compound's efficacy against the tumor-promotion stage of chemically induced carcinogenesis. An exemplary ODC assay performed with V. pahalae cell culture extracts is shown is Example 3.

In one aspect, the method for modifying the content of proanthocyanidins involves initiating the flavonoid-producing culture under conditions sufficient to initiate such culture, and establishing a pigmented cell culture under conditions sufficient to establish a pigmented culture, followed by scaling up of the pigmented cell culture for an appropriate amount of time prior to isolation of the proanthocyanidins. Accordingly, altering the conditions required to initiate a culture or establish a pigmented culture results in modified content of proanthocyanidins in such culture. Both physical aspects (e.g. irradiance) and chemical aspects (e.g. media composition) of the plant culture microenvironment may be varied to achieve the desired modified content.

For instance, sucrose concentration may be increased in a suspension medium in order to increase the amount of proanthocyanidins in the culture. Furthermore, nitrogen sources (e.g. NH₄NO₃) may be manipulated for production of secondary metabolites in plant tissue cultures (see Neera et al., Phytochemistry, 31(12):4143-4149, 1992). Therefore, decreasing the concentration of nitrogen sources in V. pahalae culture medium is believed to increase the production of proanthocyanidins in said culture. In addition, infusion of certain amino acids such as glutamine, glycine, and serine also may significantly affect the production of secondary metabolites in plant cultures. As a result, the concentration of these amino acids in V. pahalae suspension medium may be increased in order to enhance the production of proanthocyanidins. Additional amino acids can also be included in the medium and tested for their ability to modify the content of proanthocyanidins.

Lighting conditions can also be varied in order to achieve modified proanthocyanidin content in plant culture. For example, the lighting can be changed by increasing irradiance or length of exposure to the light. Additionally, the frequency or duration of subculturing periods can be prolonged in order to improve or modify the yield of proanthocyanidins. Other modifications known in the art for manipulation of plant culture microenvironments are also contemplated as being within the scope of the present invention and can be performed by one skilled in the art.

Extraction, fractionation, and identification of proanthocyanidins with modified content are performed in the same manner as described in the above sections.

The present invention also encompasses methods for performing metabolic rate/fate studies, said methods consisting of:

(a) co-incubating a flavonoid-producing cell culture with ¹⁴C-labeled precursors, thereby allowing for labeled proanthocyanidins to be produced;

(b) extracting the labeled proanthocyanidins from the flavonoid-producing cell culture;

(d) fractionating the labeled proantocyanidins by vacuum chromatography;

(c) administering a desired labeled proanthocyanidin to an animal;

(d) measuring uptake of the labeled proanthocyanidin by the cells of the animal and/or identifying metabolic products of the labeled proanthocyanidin in said animal.

Methods of initiating and establishing flavonoid-producing cell cultures are disclosed herein. Co-incubation of flavonoid-producing cultures with ¹⁴C-labeled precursors leads to the incorporation of ¹⁴C into proanthocyanidins, allowing for the production of labeled proanthocyanidins. In one embodiment, said flavonoid-producing cell culture comprises a Vaccinium cell culture, and preferably it comprises a Vaccinium pahalae cell culture. The labeled precursor can be, for example, ¹⁴C phenylalanine, but other labeled precursors known in the art can be used as well. Extraction, fractionation, and identification of labeled proanthocyanidins are performed according to the methods disclosed herein. In one embodiment of the present invention, said labeled proanthocyanidins comprise proanthocyanidin B-2, catechin, and epicatechin, and preferably, said labeled proanthocyanidin comprises proanthocyanidin B-2.

Subsequently, metabolic fate/rate studies with a desired labeled proanthocyanidin are performed in animal models. Briefly, the desired, labeled proanthocyanidin can be administered to an animal via parenteral or enteral routes. Parenteral administration includes subcutaneous, intramuscular, intradermal, intramammary, intravenous, and other administrative methods known in the art. Enteral administration includes oral, rectal, and other methods known in the art. The metabolic study factors such as mode of administration and the amount of a labeled proanthocyanidin to be administered can be easily determined and adjusted to a particular animal model by one of ordinary skill in the art.

Following the administration of the labeled proanthocyanidin to an animal, the animal is allowed sufficient time to metabolize said proanthocyanidin. Subsequently, the desired organs and/or tissues are removed in order to study the uptake of the labeled proanthocyanidin into such cells or to identify the metabolic products of said proanthocyanidin. Methods for isolating and examining organs and/or tissues are well known in the art.

Metabolic fate/rate studies can also be performed in vitro, i.e. in animal cell cultures. Briefly, following the production, isolation, and identification of labeled proanthocyanidins according to the methods described herein, a desired animal cell culture is incubated with a desired labeled proanthocyanidin for a sufficient amount of time. For the purpose of the present invention, any animal cell culture can be used. The sufficient incubation time will vary depending on the experiments performed, and can easily be determined by one of ordinary skill in the art. Following the incubation, the uptake of the labeled proanthocyanidin by the cells is measured, and/or metabolic products of the labeled proanthocyanidin in said cells are identified.

A preferred way to measure the uptake is by liquid scintillation counting. For example, the isolated cells are lysed, and the samples of the lysates, the medium, and the washes of the cell monolayer are subjected to the counting assay. The data from this assay would provide information as to how much of the labeled proanthocyanidin was taken up by the cells. For structurally characterizing the metabolic products of the labeled proanthocyanidin, HPLC and LC/MS are performed. The identification of the metabolic products is preferably performed by mass spectrometry analysis.

These above-mentioned assays are well known to one skilled in the art. A detailed example on how to perform metabolic rate/fate studies is provided by Boulton et al., 1999. This publication addresses the uptake and metabolism of the flavonoid quercetin, however a skilled artisan would be able to make necessary adjustments to adapt the technique to a desired proanthocyanidin.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

The following examples illustrate the invention, but are not to be taken as limiting the various aspects of the invention so illustrated.

### EXAMPLES

### Example 1

Extraction and Fractionation of Proanthocyanidins. Callus and suspension cultures were initiated from stable, continuous shoot microcultures of V. pahalae according to protocols established by Smith et al. (1997). Uniform, unpigmented suspensions were maintained by routinely transferring 3.5 ml packed cell volume to 80 ml fresh maintenance suspension medium in 250 ml flasks, at 7 d intervals, and incubating on a rotary shaker at 150 ml rpm in the dark. The suspension medium was composed of Woody Plant Medium major and minor salts (Lloyd and McCown, 1980), rose vitamins (Rogers and Smith, 1992), and 30 g l⁻¹ sucrose, 0.1 g l^{31 1} polyvinylpyrrolidone, 4.5 μM 2,4-dichloroacteic acid (2,4-D), 5.4 μM naphthaleneacetic acid, and 4.6 μM kinetin.

Pigmented suspension cultures were established by transferring 4.0-6.0 ml packed cell volume from dark-incubated cell suspensions into 35 ml of color-induction suspension medium in 125 ml flasks, and incubating under 100 μmol⁻² s⁻¹ irradiance provided by cool white fluorescent lamps. Color-induction medium differed from the medium used for dark-grown suspensions in that it contained 50 g l⁻¹ sucrose, and 20 μM benzyladenine was substituted for the kinetin. Cell cultures were subcultured at 12-14 d intervals, and cultures were maintained for four or five subculture cycles prior to use in these experiments.

Extracts were prepared in a similar manner from frozen fruits [American elderberry, Sambucus canadensis L.; American cranberry, Vaccinium macrocarpon Ait. var. Howes; chokeberry, Aronia melanocarpa (Michx.) Ell.], and powdered grape skin pigment and grapeseed extracts (Vitis vinfera L.). Frozen cranberries were provided by Decas Cranberry Co., Carver, Mass., and frozen chokeberries were provided by Artemis International, Inc., Fort Wayne, Ind. Elderberries were collected locally. Grape skin and grapeseed extracts (Traconol) were supplied by Traco Labs, Inc., Champaign, Ill.

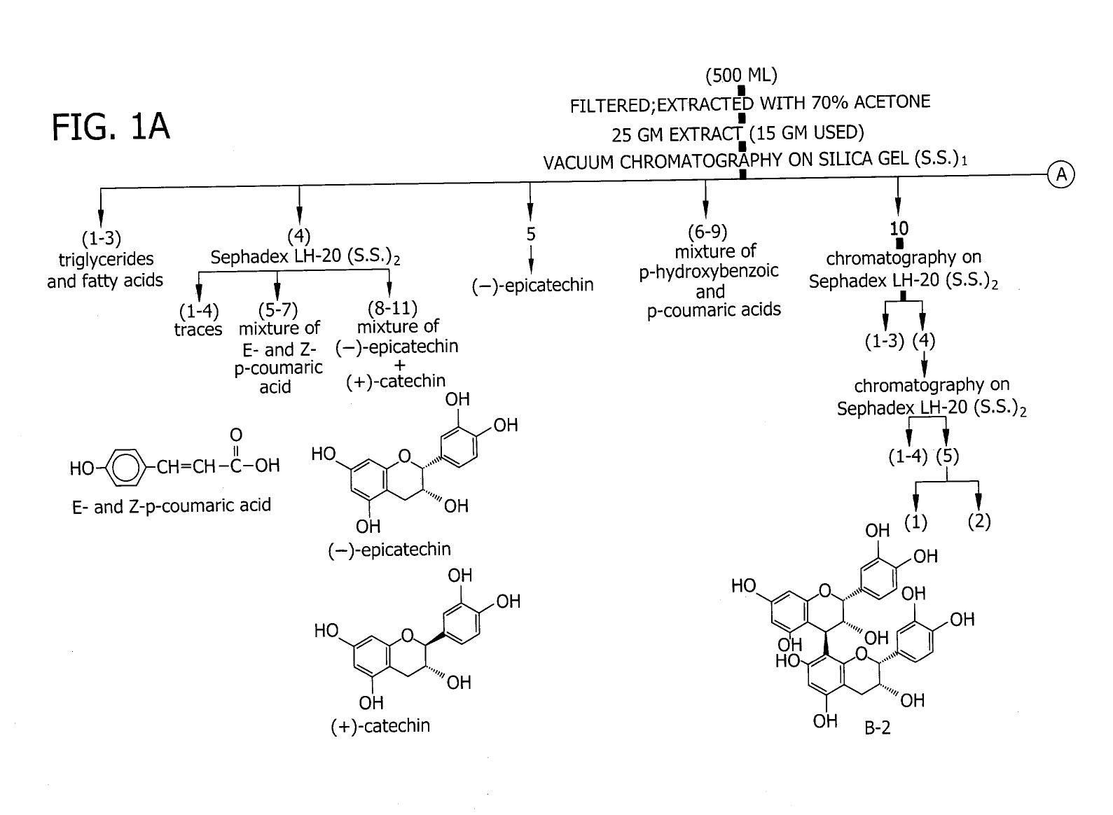

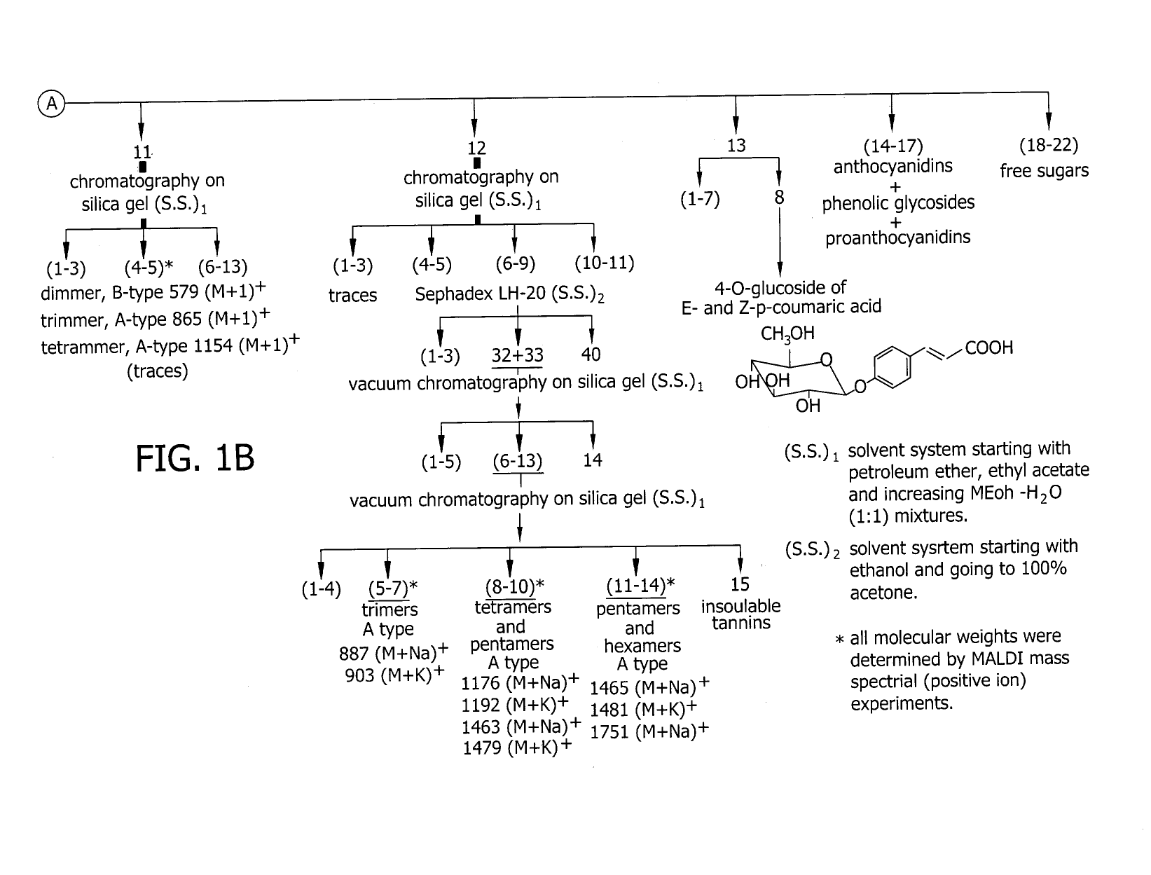

Cell cultures (approx. 500 ml volumes) were filtered and extracted with 70% acetone. The acetone was removed under vacuum and the remaining aqueous solution frozen and freeze-dried to yield a red solid (25 g). Samples of the extract (15 g) were mixed with silica gel 60 (5 g), air-dried, and the resulting mixture loaded on a column with silica gel 60 (50 g) that had been washed with petroleum ether (total 210 ml). The column was again washed with petroleum ether (150 ml). Subsequent fractions were eluted and with ethyl acetate (solvent A); increasing amounts of methanol-water (1:1, solvent B) were added up to 100% B. At this point, all colored materials were removed from the column, and 22 fractions were collected (FIG. 1). These fractions were concentrated under vacuum to remove volatile solvents, and water was removed from the remaining portion of each sample by lyophilization.

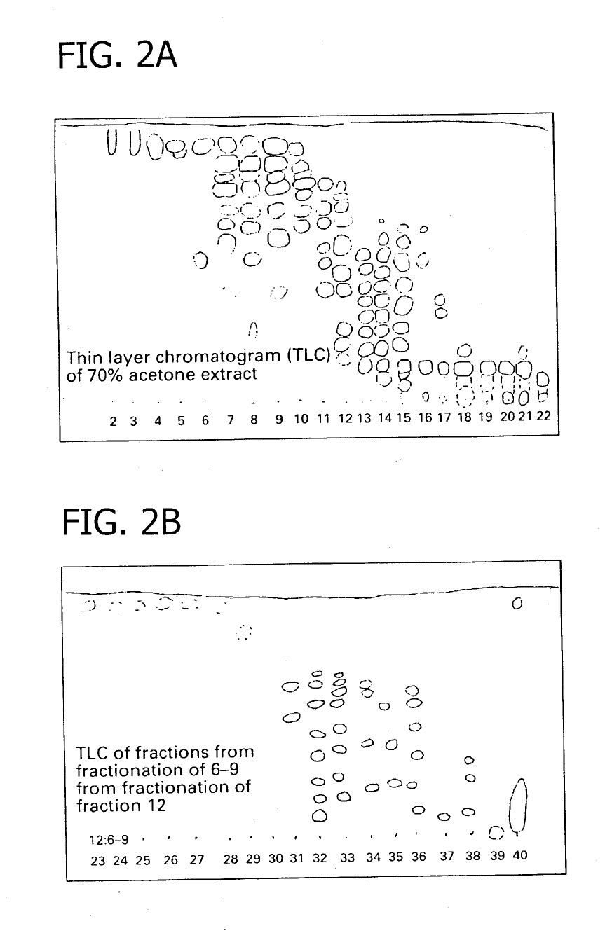

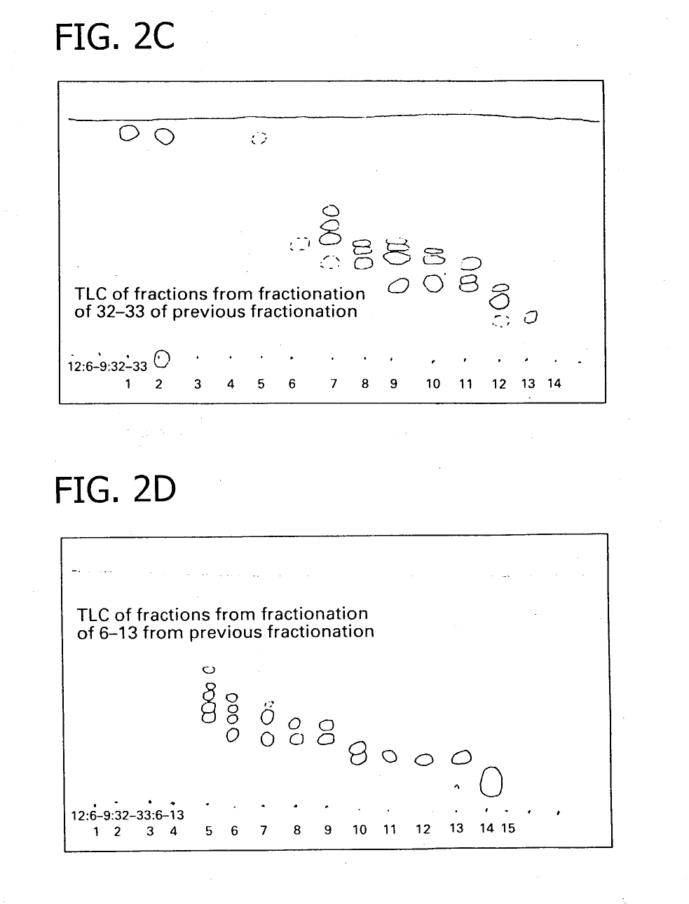

Several of the resulting fractions (1-22) were fractionated by additional chromatography on either silica gel or Sephadex LH-20. Each step of this fractionation was monitored by TLC on silica gel plates with ethyl acetate-methanol-water (79:11:10), using vanillin-HCl reagent and with dichromate solution followed by heating at 100° C. in each instance. Another plate was sprayed with FeCl₃ reagent. Further fractionation and purification of subfractions was accomplished by repeating the procedure, but varying solvent composition to achieve optimal separations (FIGS. 1 and 2).

The series of fractionations leading to isolation of purified compounds such as proanthocyanidin B-2 and simplified mixtures of other proanthocyanidins is depicted in FIG. 1. The TLC of the 70% acetone extract (fractions 1-22) is shown in FIG. 2a. In the fractionation series (FIG. 1), fraction 12 was then fractionated to yield a series of fractions. Of these, fractions 6-9 were combined and again fractionated by chromatography on silica gel (FIG. 1). The fractions from this procedure were then subjected to further TLC (FIG. 2b). Fractions 32 and 33 were combined and again fractionated by chromatography on silica gel (FIG. 1). The TLC of the resulting fractions is shown in FIG. 2c. Fractions 6-13 were then recombined and again fractionated by chromatography on silica gel (FIG. 1). The TLC of the fractions from this separation is shown in FIG. 2d.

In FIG. 2a, the presence of dark blue spots in all except the first three fractions indicated the presence of phenolic materials. Visualization of a similar TLC plate with vanillin-HCl reagent followed by heating indicated the presence of monomeric flavan-3-ols or proanthocyanidins (pink-red color) in fractions 4-20.

The presence of peaks in ¹H-NMR spectra at 5.2 (m), 4.19 (q), 4.21 (dd), 2.2 (t), 2.1 (m), 1.5 (m), 1.3 (s), and 0.9 (t) is typical for fatty acids of triacylglycerides as seen in fractions 1-3 (Chen et al., 1999). Fraction 4, which contains acidic materials with an R_{f} value of 0.9, was fractionated to yield subfractions consisting of a mixture of E- and Z-p-coumaric acid, probable fatty acids (R_{f}=0.9), mixtures of (−)-epicatechin and (+)-catechin, and more polar proanthocyanidins. Absorptions in the ¹H-NMR spectra of fraction 6 at δ6.2 (d, J=16 Hz) for (—HC═CH—), 6.8 (d, J=8.5 Hz), and 7.42 (d, J=8.5 Hz) indicate the presence of a p-coumarate moiety. Other smaller peaks suggest that p-hydroxybenzoate compounds also may be present. Peaks at 178.590 (probable C═O); 159.884 (C-4); 144.409 (HC═CH-phenyl); 130.366 (C-2,6); 124.7 (C-1); 116.638 (C-3,5); and 115.674 ppm (CH═CH-phenyl) in the ¹³C-NMR spectrum indicate the presence of a more polar p-hydroxybenzoate or a p-hydroxycoumarate moiety.

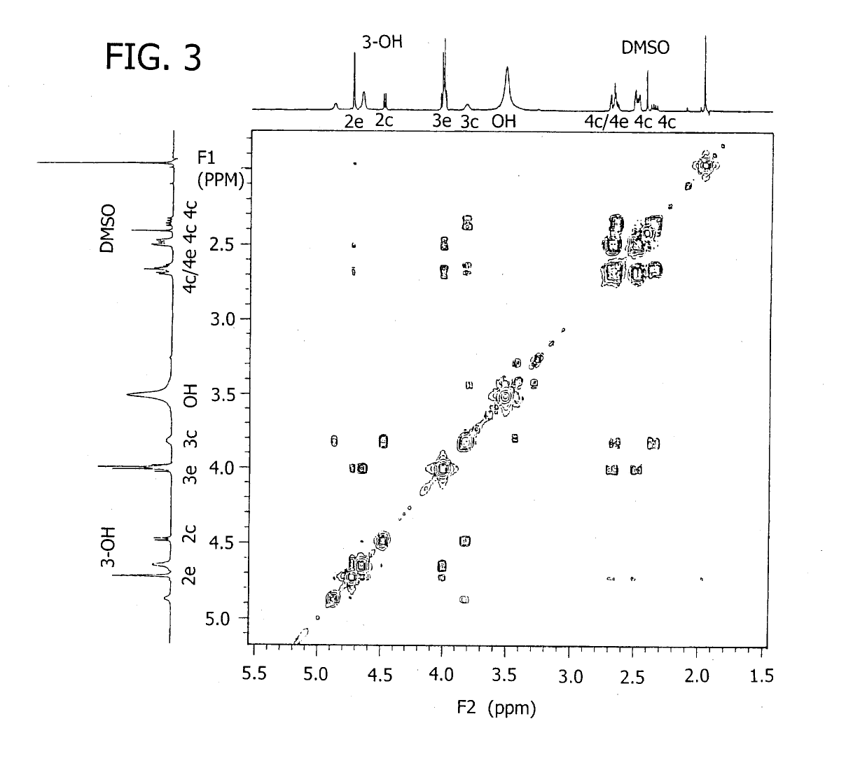

(−)-Epicatechin and (+)-catechin (R_{f}=0.81-0.89) are major components of fractions 4-6 and are also found in smaller amounts in fractions 7-10. These two flavan-3-ols occur in an approximate 6:1 ratio as judged by both the ¹H- and ¹³C-NMR spectra. The assignment of protons in these compounds was facilitated by determination of a correlation spectroscopy spectrum (FIG. 3). Proton 2 of (−)-epicatechin (4.74δ, ax) is coupled to proton 3 at 4. 4.01 (eq). The signal appears as a relatively sharp singlet (J<1 Hz). Proton 3 is, in turn, coupled to both protons at position 4 (2.5 and 2.79). The proton couplings of the minor compound, (+)-catechin, are also clearly resolved. Proton 2 (4.49) is coupled to proton 3 at 3.83, but in this instance is a doublet (J=7.4 Hz). Proton 3 is again seen to be coupled to both protons at position 4 (2.4 and 2.7). Each of these protons at position 4 is coupled to the other in both compounds. The chemical shifts and coupling constants of these compounds were similar to those previously reported (Thomson et al., 1972; Jacques et al., 1974; Fletcher et al., 1977; Morimoto et al., 1986; Kashiwada et al., 1990). The FAB mass spectrum had a parent ion of 291.1 m/z (M+1)¹ (molecular weight of catechin and epicatechin, 290.33).

Proanthocyanidin B-2 is a component of fractions 7-10, 12 and 13 (R_{f}=0.67-0.70; FIGS. 1 and 2). Fractions containing this dimer were further purified by chromatography on Sephadex LH-20 gel with 70% ethanol. Both the ¹H-NMR and ¹³C-NMR data of the isolate were identical to those previously reported for proanthocyanidin B-2 (Thomson et al., 1972; Jacques et al., 1974; Fletcher et al., 1977; Morimoto et al., 1986). Additionally, the positive-ion FAB and MALDI mass spectra for this compound had parent ions at 579 m/z (M+1)⁺, corresponding to that expected for a proanthocyanidin dimer.

The 4-O-glucosides of E- and Z-p-coumaric acid (R_{f}=0.25-0.30) were found in fractions 12 and 13. These compounds can be readily detected by use of bromcresol green for visualization of the TLC plates, as they produce bright yellow spots. A mixture of the two glucosides was isolated by additional fractionation. Fraction 12 was selected for subfractionation because it contained a large number of proanthocyanidins, some of which had R_{f} values similar to those with activity in the ODC bioassay in previous studies with blueberry (Bomser et al., 1996).

In addition to the compounds above, TLC revealed a series of phenolic compounds of increasing polarity that occur in fractions 4-22, which were obtained by vacuum chromatography of the 70% acetone extract on silica gel (FIGS. 1 and 2). Based on this TLC, there appeared to be about 20 total flavan-3-ols and proanthocyanidins; at least 14 occurred in fraction 12, the most diverse fraction assuming that each TLC spot represents only one compound. Fractions 6-18 appeared to consist primarily of flavan-3-ols and proanthocyanidins, as indicated by TLC followed by visualization with FeCl₃ and vanillin-HCl reagents.

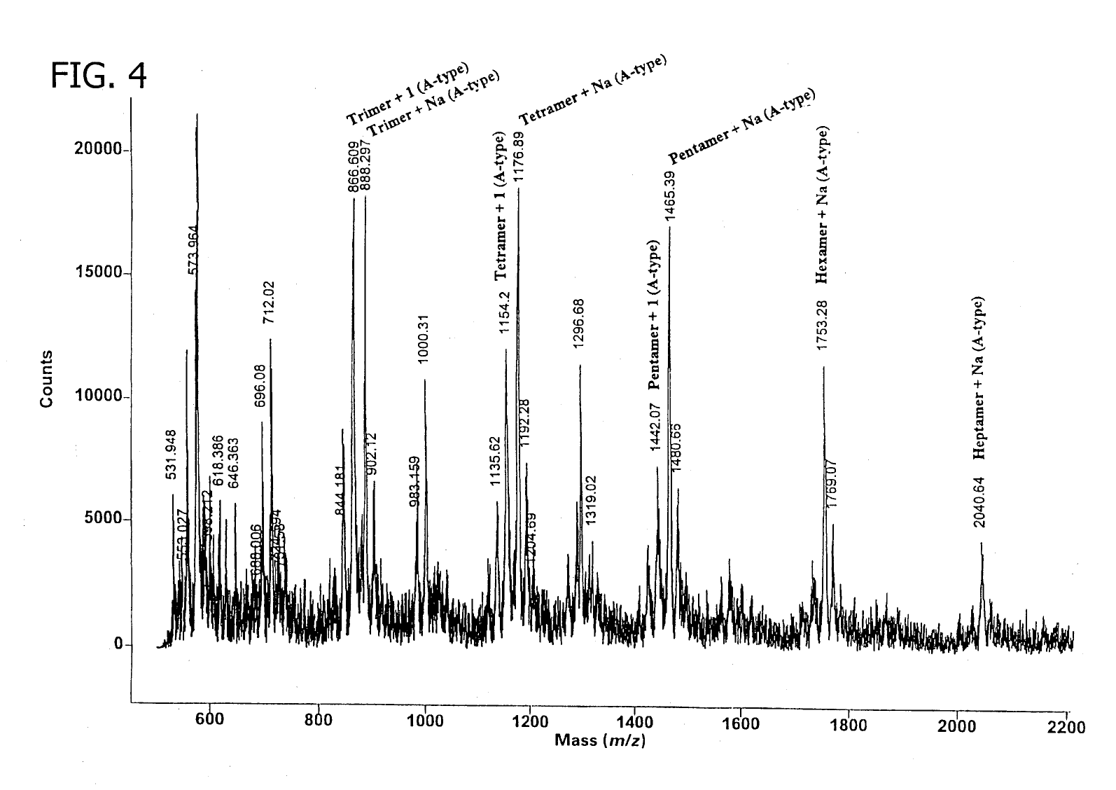

In the positive-ion FAB mass spectra of subfraction 4 of fraction 11, and subfraction 4 of fraction 12, significant peaks corresponded to the presence of trimers and tetramers containing one A-type linkage {865.3 [M+1]³⁰ (trimer, B-type), and 1153.4 m/z [M+1]⁺(tetramer, A-type)}, in addition to those for a dimer of two epicatechin or catechin units (579.2 m/z). The positive ion mass spectrum of subfraction 8 of fraction 12 (FIG. 4) had peaks corresponding to an A-type trimer [M+1]+(866.609), [trimer+Na]+(888.297); [tetramer+1]+(1154.2), [tetramer+Na]⁺(1465.39), [hexamer+Na]+(1753.28), and [heptamer+Na]+(2040.64).

In addition to proanthocyanidins, anthocyanins also were present in fractions 13-18. These compounds were removed in subsequent fractionation steps. Although most of the individual proanthocyanidins had not been isolated, purified, and characterized, ¹H-NMR, ¹³C-NMR, and mass spectra of the fractions indicated the presence of a mixture of flavan-3-ols (such as catechin and epicatechin) and proanthocyanidin dimers and higher oligomers.

Some evidence for structurally modified compounds was seen in certain fractions, for example fraction 10 subfraction 4, where a peak at 758.7 m/z in the mas spectrum corresponded in molecular weight to cinchonain IIa or IIb (Nonaka et al., 1982), or possibly a p-coumaryl ester of a dimeric proanthocyanidin.

Thus, V. pahalae cell culture contains (+)-catechin, (−)-epicatechin, proanthocyanidin B-2, a series of other proanthocyanidins ranging from dimers to heptamers, as well as a mixture of E- and Z-p-coumaric acid, the corresponding 4-O-glucoside, and other derivatives containing E- and Z-p-coumaric acid derivatives. Although not all compounds were completely purified or characterized, single entities and mixtures of proanthocyanidins were obtained by this relatively straightforward procedure. It was shown in this example that the initial extract could be fractionated successfully on silica gel to yield a series of fractions containing mixtures of increasingly polar proanthocyanidins (FIGS. 2a-d).

### Example 2

Galvinoxyl free radical quenching assay. An antioxidant assay originally reported by Smith and Hargis (1985) was adapted for examination of phenolic antioxidants (see Smith et al., 2000). Galvinoxyl, a stable free radical, was obtained from Sigma. All solvents, including water, were glass-distilled. A solution of galvinoxyl in methanol was prepared that had an initial absorbance of 1.8-2.0 OD at 429 nm. As the compound slowly reacts with oxygen, or is reduced via electron transfer, the chromophore is lost and its absorbance decreases. The loss of absorbance was monitored over time to give a rate constant for reactivity with a good electron donor (the reaction with oxygen is extremely slow, but is always monitored as a control value to ensure that rapid absorbance loss is due to the added antioxidant). Quenching of the galvinoxyl radical was recorded for 5 minutes at 30 s intervals using a Beckman DU 7400 spectrophotometer with a scan rate of 0.5 s. Aliquots of extracts from cell cultures, or from fruits and fruit preparations (for comparison) were dissolved in methanol or water at an initial concentration of 10 mg ml⁻¹. These solutions were prepared fresh daily, stored at 0° C. in the dark and diluted as necessary. An aliquot (0.1 ml) of the dissolved fruit or cell culture extracts (concentrations ranging from 0.1-10 mg ml⁻¹) was added to a galvinoxyl solution (2 ml), so that the final concentration of the extract/fraction was 5-500 μg ml⁻¹. Data were then plotted as a function of ln(Abs t/Abs 0) versus time to obtain rate plots, the slope of which yielded k, the rate constant. Rate constants were calculated using first-order kinetics based on decrease of absorbance versus time. At least three concentrations of each extract/preparation were measure in order to obtain regression coefficients that confirmed first-order rate constants. EC_{first} is the lowest effective concentration needed to quench the galvinoxyl radical following first-order kinetics.

Ohelo cell culture extract was tested in tandem with extracts from frozen fruits, powdered juice or seed preparations. In general, in galvinoxyl free radical quenching assay, the most effective antioxidants provide a relatively low half-life value (t_{1/2} min) at the lowest possible effective concentration needed to quench the galvinoxyl radical (EC_{first}). Linear data, following first-order kinetics, were obtained at 500 μg ml⁻¹ for cranberry, and at 50 μg ml⁻¹ for other fruits, which permitted direct comparisons between fractions. For ohelo cell culture and grapeseed extracts, rapid quenching of the galvanoxyl radical was achieved at lower concentrations (5.0 and 0.5 μg ml⁻¹, respectively). A comparison of the efficacy of each source of extracts as antioxidants is presented in Table 1. The most powerful antioxidant capacity in this assay was exhibited by the grapeseed extract (Traconol), which is marketed on the basis of its oligomeric and polymeric proanthocyanidin content. The ohelo cell culture extract was clearly more effective as a free-radical quencher at lower concentrations than any of the other fruit extracts tested (Table 1).

### Example 3

Ornithine decarboxylase assay. Mouse epidermal cells, line 308, were grown at 37° C. in humidified incubators containing 5% CO₂ in air. Minimal essential medium, spinner modification (S-MEM), supplemented with 5% dialyzed fetal bovine serum, non-essential amino acids (1×), Ca⁺² (0.05 mM), and antimycotic-antibiotic (1%), was used as the growth medium and was replaced three times per week (Lichti and Gottesman, 1982). 90% confluent cells were washed with Ca⁺²- and Mg⁺²-free Dulbecco's PBS, refed with growth medium, allowed to grow for an additional 24 h, then plated at 2×10⁵ cells ml⁻¹ per well in 24-well plates. Plates were placed in an incubator (37° C., 5% CO₂) for 18 h, after which time 5 μl of sample (in DMSO), and 20 μl of 12-O-tetradecanoylphorbol-13-acetate (TPA) solution (final 200 nM, dissolved in 2.5% DMSO) were added to each well. Cells were incubated for an additional 6 h, washed twice with cold Ca⁺²-, Mg⁺²-free PBS, then immediately placed in a −80° C. freezer until the ornithine decarboxylase (ODC) assay was performed, usually within 3 days.

Two sets of experimental controls were used for this assay: one set of additional wells did not receive any culture extract, only an equivalent amount of DMSO (0.6%); a second set was DMSO- and TPA-treated. Ornithine decarboxylase activity was determined by measuring the release of ¹⁴CO₂ from L-[1-¹⁴C] ornithine essentially by the procedure of Lichti and Gottesman (1982), as described previously (Gerhauser et al., 1995). The protein content of each of the 24 wells used for the ODC assay was determined following the addition of chloramine T (50 μl, 5.7 N) to solubilize protein (Higuchi and Yoshida, 1977). TPA-induced ODC activity was expressed as cpm ¹⁴CO₂ released mg⁻¹ protein h⁻¹, and the data are expressed as a percentage relative to the sample treated with TPA, after subtracting the DMSO group. The amount of fraction required to inhibit ODC activity by 50% (IC₅₀) was determined graphically from quadruplicate measurements.

ODC activity is greatly and rapidly induced in response to growth-promoting stimuli such as growth factors, hormones, and tumor promoters. One intensely studied inducer of ODC activity is the tumor promoter TPA, and this Example is based on inhibition of TPA-induced ODC activity.

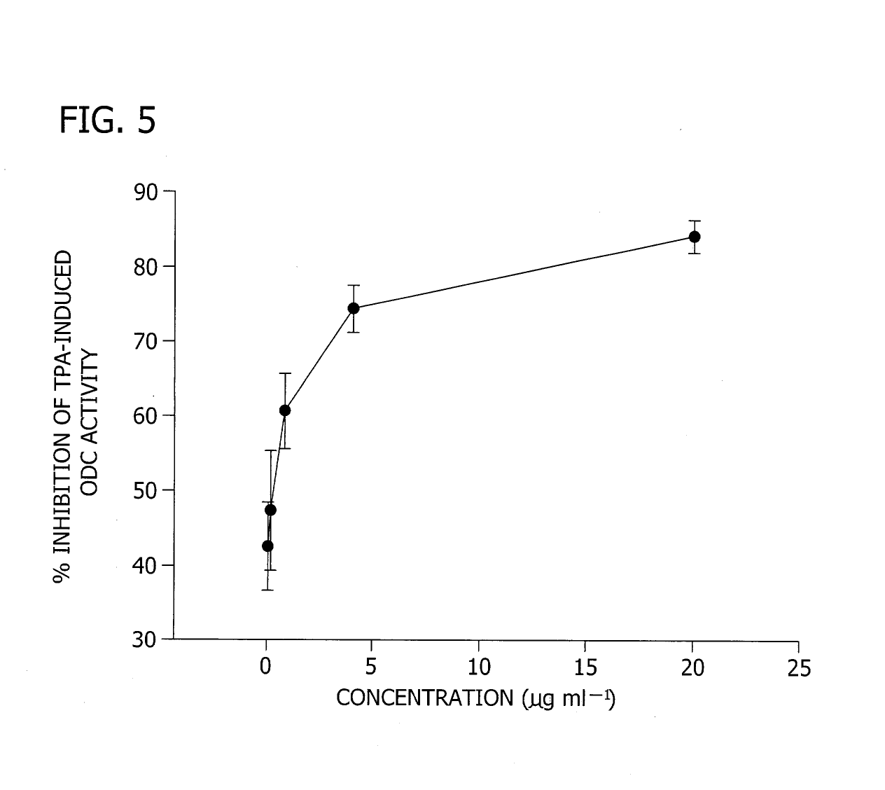

In the ODC assay with the ME-308 cell line, the IC₅₀ value for the ohelo cell culture was 0.24 μg ml⁻¹, which was indicative of significant activity against the promotion stage of chemically induced carcinogenesis. Ornithine decarboxylase activity of DMSO-treated controls and TPA-+DMSO-treated controls were 163 and 1874 nmol mg⁻¹ protein h⁻¹, respectively. The amounts of ohelo cell culture extract needed to provide significant inhibition in the ODC assay were not associated with cytotoxicity. Based on the results from more than 1000 natural plant extracts evaluated for putative bioactivity, an extract is determined as active when the IC₅₀ value is equal to or lower than 4 μg ml⁻¹ in the ODC assay system (Pezzuto, 1995). Accordingly, 0.24 μg ml⁻¹ value obtained for ohelo cell culture extracts was considered highly significant (FIG. 5).

What is claimed is:

1. A method for isolation of proanthocyanidins from a flavonoid-producing cell culture, said method comprising the steps of:
(a) initiating the flavonoid-producing cell culture;
(b) establishing a pigmented cell culture by utilizing the culture from (a);
(c) extracting the proanthocyanidins from the pigmented cell culture; and
(d) fractionating the proanthocyanidins by vacuum chromatography.

2. The method of claim 1, further comprising the step of identifying the proanthocyanidins.

3. The method of claim 2, wherein said identification step comprises performing ¹H-NMR, ¹³C-NMR, or MS.

4. The method of claim 2, wherein the proanthocyanidins comprise proanthocyanidin B-2, catechin, or epicatechin.

5. The method of claim 4, wherein the proanthocyanidin is proanthocyanidin B-2.

6. The method of claim 1, wherein the flavonoid-producing cell culture comprises a Vaccinium cell culture.

7. The method of claim 6, wherein the Vaccinium cell culture is a Vaccinium pahalae cell culture.

8. The method of claim 1, said method further comprising varying conditions for initiating and establishing the cell cultures of (a) and (b) in a manner so as to modify the content of proanthocyanidins in the flavonoid-producing cell culture.

9. A method for isolation of proanthocyanidins from a Vaccinium pahalae cell culture, said method comprising the steps of:
(a) initiating the Vaccinium pahalae culture;
(b) establishing a pigmented cell culture by utilizing the culture from (a);
(c) extracting the proanthocyanidins from the pigmented cell culture;
(d) fractionating the proanthocyanidins by vacuum chromatography; and
(e) identifying the proanthocyanidins by performing one or more of ¹H-NMR, ¹³C-NMR or MS.

10. A method of modifying the content of proanthocyanidins in a flavonoid-producing cell culture, said method comprising:
(a) initiating the flavonoid-producing cell culture under conditions sufficient to initiate said culture;
(b) establishing a pigmented cell culture by utilizing the culture from (a) under conditions sufficient to establish the pigmented culture; and
(c) expanding the pigmented cell culture for an appropriate amount of time prior to isolation of the proanthocyanidins.

11. The method of claim 10, wherein the conditions from (a) and/or (b) are varied in a manner so as to increase the content of proanthocyanidins in the flavonoid-producing cell culture.

12. The method of claim 10, wherein modifying the content of the proanthocyanidins in the flavonoid-producing cell culture increases the anti-oxidant capacity of said proanthocyanidins.

13. The method of claim 10, wherein modifying the content of the proanthocyanidins in the flavonoid-producing cell culture increases the anti-carcinogenic capacity of said proanthocyanidins.

14. The method of claim 10, wherein the flavonoid-producing culture comprises a Vaccinium cell culture.

15. The method of claim 14, wherein the Vaccinium cell culture is a Vaccinium pahalae cell culture.

16. The method of claim 10, wherein the isolation of the proanthocyanidins comprises purification of proanthocyanidin B-2, catechin, and epicatechin.

17. The method of claim 16, wherein said purification comprises the purification of proanthocyanidin B-2.

18. A method of performing metabolic rate/fate studies, said method comprising the steps of:
(a) co-incubating a flavonoid-producing cell culture with ¹⁴C-labeled precursors, thereby allowing for labeled proanthocyanidins to be produced;
(b) extracting the labeled proanthocyanidins from the flavonoid-producing cell culture;
(d) fractionating the labeled proanthocyanidins by vacuum chromatography;
(c) administering a desired labeled proanthocyanidin to an animal; and
(d) measuring uptake of the labeled proanthocyanidin by the organs and/or tissues of the animal and/or identifying metabolic products of the labeled proanthocyanidin in said animal.

19. The method of claim 18, wherein the step of measuring the uptake of the labeled proanthocyanidin comprises liquid scintillation counting.

20. The method of claim 18, wherein the step of identifying the metabolic products of the labeled proanthocyanidin comprises mass spectrometry analysis.

21. The method of claim 18, wherein the desired proanthocyanidin is selected from the group consisting of proanthocyanidin B-2, catechin, and epicatechin.

22. The method of claim 21, wherein the desired proanthocyanidin is proanthocyanidin B-2.

23. The method of claim 18, wherein the flavonoid-producing cell culture comprises a Vaccinium cell culture.

24. The method of claim 23, wherein the Vaccinium cell culture is a Vaccinium pahalae cell culture.

25. A method of performing metabolic rate/fate studies, said method comprising the steps of:
(a) co-incubating Vaccinium pahalae cell culture with ¹⁴C-labeled precursors, thereby allowing for labeled proanthocyanidins to be produced;
(b) extracting the labeled proanthocyanidins from the Vaccinium pahalae cell culture;
(d) fractionating the labeled proanthocyanidins by vacuum chromatography;
(c) administering a desired labeled proanthocyanidin to an animal; and
(d) measuring uptake of the labeled proanthocyanidin by the organs and/or tissues of the animal and/or identifying metabolic products of the labeled proanthocyanidin in said animal.

26. A method of performing metabolic rate/fate studies, said method comprising the steps of:
(a) co-incubating a flavonoid-producing cell culture with ¹⁴C-labeled precursors, thereby allowing for labeled proanthocyanidins to be produced;
(b) extracting the labeled proanthocyanidins from the flavonoid-producing cell culture;
(d) fractionating the labeled proanthocyanidins by vacuum chromatography;
(c) incubating an animal cell culture with a desired labeled proanthocyanidin; and
(d) measuring uptake of the labeled proanthocyanidin by cells in the animal cell culture and/or identifying metabolic products of the labeled proanthocyanidin in said cells.

27. The method of claim 26, wherein the step of measuring the uptake of the labeled proanthocyanidin comprises liquid scintillation counting.

28. The method of claim 26, wherein the step of identifying the metabolic products of the labeled proanthocyanidin comprises mass spectrometry analysis.

29. The method of claim 26, wherein the desired proanthocyanidin is selected from the group consisting of proanthocyanidin B-2, catechin, and epicatechin.

30. The method of claim 29, wherein the desired proanthocyanidin is proanthocyanidin B-2.

31. The method of claim 26, wherein the flavonoid-producing cell culture comprises a Vaccinium cell culture.

32. The method of claim 31, wherein the Vaccinium cell culture is a Vaccinium pahalae cell culture.

33. A method of performing metabolic rate/fate studies, said method comprising the steps of:
(a) co-incubating a Vaccinium pahalae cell culture with ¹⁴C-labeled precursors, thereby allowing for labeled proanthocyanidins to be produced;
(b) extracting the labeled proanthocyanidins from the Vaccinium pahalae cell culture;
(d) fractionating the labeled proanthocyanidins by vacuum chromatography;
(c) incubating an animal cell culture with a desired labeled proanthocyanidin; and
(d) measuring uptake of the labeled proanthocyanidin by cells in the animal cell culture and/or identifying metabolic products of the labeled proanthocyanidin in said cells.
